(19) United States
(12) Patent Application Publication
(10) Pub. No.: US 2011/0008412 A1
(43) Pub. Date: Jan. 13, 2011

(54) Oral Formulations for 5-HT-Receptor Agonists, Uses and Methods of Treatment Employing The Same

(75) Inventors: Amar LULLA, Mumbai (IN); Geena MALHOTRA, Mumbai (IN)

Correspondence Address: CONLEY ROSE, P.C., 5601 GRANITE PARKWAY, SUITE 750, PLANO, TX, 75024, US

(73) Assignee: CIPLA LIMITED, Mumbai (IN)

(21) Appl. No.: 12/887,599

(22) Filed: Sep. 22, 2010

Related U.S. Application Data
(63) Continuation of application No. 10/577,760, filed on Dec. 18, 2006, now abandoned.

(30) Foreign Application Priority Data
Oct. 30, 2003 (GB) 0325383.8

(51) Int. Cl. A61K 9/70; A61K 31/4045; A61K 9/42; A61K 9/16; A61P 25/06

(52) U.S. Cl. 424/443; 514/415; 424/476; 424/498

(57) ABSTRACT

A pharmaceutically acceptable oral formulation comprising core material which comprises a therapeutically effective amount of a 5-HT-receptor agonist, or a pharmaceutically acceptable salt, solvate or derivative thereof, which core material is provided with a substantially water resistant coating comprising one or more substantially water resistant materials.

### CROSS-REFERENCE TO RELATED APPLICATIONS

This is a Continuation Application of U.S. patent application Ser. No. 10/577,760 having a 371(c) date of Dec. 18, 2006 and published as US 2007/0077299 A1 and entitled “Oral Formulations for 5-HT-Receptor Agonists, Uses and Methods of Treatment Employing the Same,” which was a filing under 35 U.S.C. 371 of International Application No. PCT/GB2004/004605 filed Nov. 1, 2004, entitled “Oral Formulations For 5-HT-Receptor Agonists, Uses And Methods Of Treatment Employing The Same,” claiming priority of Great Britain Patent Application No. GB 0325383.8 filed Oct. 30, 2003, which applications are incorporated by reference herein in their entirety.

### FIELD OF THE INVENTION

The present invention is concerned with a pharmaceutically acceptable oral formulation comprising a 5-HT-receptor agonist, in particular sumatriptan, a process for preparing such a formulation, therapeutic uses thereof and methods of treatment employing the same, and also uses of one or more waxes, or one or more wax derivatives, in inhibiting degradation of a 5-HT-receptor agonist.

### BACKGROUND OF THE INVENTION

Serotonin agonists, also known as 5-HT-receptor agonists or 5-HT_{1D}-receptor-selective agonists, have unique properties that result in constriction of intracranial blood vessels. Sumatriptan was first in the series of new serotonin-receptor agonists available for the treatment of acute migraine attacks. Other such agents for the acute treatment of migraine now also include zolmitriptan, naratriptan and rizatriptan.

Migraine headache afflicts 10% to 20% of the population. The frequency of migraine attacks is extremely variable, but usually ranges from one to two per year to one to four per month. The efficacy of antimigraine drugs varies with undefined environmental and genetic factors. A rather vague and inconsistent pathophysiological characteristic of migraine is the spreading depression of neural impulses from a focal point of vasoconstriction, followed by vasodilatation. The literature reports that 5-HT is a key mediator in the pathogenesis of migraine, and as such 5-HT-receptor agonists have become the mainstay for acute treatment of migraine headaches.

The introduction of 5-HT-receptor agonists, such as sumatriptan, zolmitriptan, naratriptan, rizatriptan and the like, which are also generically known as triptans, in the therapy of migraine has led to significant progress in preclinical and clinical research relating to migraine. At the scientific level, the selective pharmacological effects of these agents, referred to as triptans, at 5-HT receptors have led to new insights into the pathophysiology of migraine. At the clinical level, the drugs are effective, acute antimigraine agents. Their ability to decrease, rather than exacerbate, the nausea and vomiting of migraine is also an important advance in the treatment of the condition.

The triptans are derivatives of indole, with substituents on the 3 and 5 positions. Sumatriptan, 3-[2-(dimethylamino)ethyl]-N-methyl-1H-indole-5-methanesulfonamide, is widely employed in the form of its succinate salt, namely 3-[2-(dimethylamino)ethyl]-N-methyl-1H-indole-5-methanesulfonamide succinate. Sumatriptan has the following structural formula

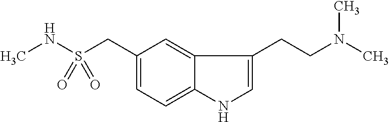

Sumatriptan is an agonist for a vascular 5-HT₁ receptor subtype, a member of the 5-HT_{1D} family. The vascular 5-HT₁ receptor subtype that sumatriptan activates is present on the human basilar artery, and in the vasculature of human dura mater and mediates vasoconstriction. This action in humans correlates with the relief of migraine headache.

Several formulations of 5-HT receptor agonists have been reported in the literature, many of which relate to formulations of sumatriptan. For example, formulations relating to effervescent, oral, transmucosal, fast dispersing, disintegrating, controlled release and pulse release compositions for sumatriptan have been reported. Examples of patents describing such formulations are as follows.

GB 2262445B covers a pulsed release dosage form, which provides an immediate dose of sumatriptan followed by a further dose after a time delay of 1 to 6 hours. GB 2262445B also describes a process for preparing a tablet, wherein the tablet core is further coated by a dry powder coat by compression.

GB 2162522B also describes film coated tablet formulations of sumatriptan succinate.

WO 02/083219 describes a dispensing apparatus for dispensing a unit dose unit of sumatriptan, in particular for intranasal administration. The unit dose is contained in a cylinder, which is moved relative to a piston to expel the contents thereof through a passage in the piston and out of a nozzle opening.

US 2003/0021755 describes delivery of antimigraine compounds through an inhalation route. More particularly, the specification relates to condensation aerosol formulations to be inhaled and which comprise sumatriptan, frovatriptan, naratriptan or the like.

GB 2254784B describes a pharmaceutical composition of sumatriptan for oral administration, comprising a film-coated solid dosage form. The film-coated solid dosage forms are of use in the treatment of conditions associated with cephalic pain, in particular migraine. GB 2254784B also describes that the unpleasant taste associated with oral administration of sumatriptan is substantially eliminated by the formulations described therein, and more particularly by the film coating. Furthermore, the film coating makes the formulations easier to handle and reduces potentially hazardous dust formation occurring during the packaging or administration of the drug. The film coating comprises suitable polymers.

U.S. Pat. No. 5,807,571 describes a transdermal therapeutic system for the systemic administration of sumatriptan. The system can be advantageous as the half-life of sumatriptan after subcutaneous and oral application merely amounts to about 2 hours. The bioavailability in case of oral application merely amounts to 14% due to the presystemic metabolism, while it amounts to 96% when injected subcutaneously. Owing to the short half-life of sumatriptan, migraine symptoms can soon return, requiring new application. Furthermore, when sumatriptan is injected, side effects may occur as a burning and redness at the puncture point. Also, a temporary sensation of heat, pressure, narrowness or heaviness is generally observed after the application of sumatriptan.

WO 94/26270 also describes a transdermal therapeutic system for the systemic administration of sumatriptan.

It will be appreciated from the prior art discussed above that many different formulations for antimigraine compounds for oral and systemic administration have been described in the prior art. Oral formulations of antimigraine compounds have to date been most popular, in view of advantages associated with the use thereof, for example convenience of use, lower cost, ease of availability and the like.

There are, however, certain disadvantages associated with known oral dosage forms of antimigraine agents and in particular it would be desirable to provide a pharmaceutically acceptable solid oral formulation, which would lessen or substantially prevent the possible degradation of antimigraine compounds in the presence of moisture. More particularly, it would be advantageous to provide a formulation which could alleviate the effects of contact of ambient air and moist environment on known antimigraine compounds. We have now surprisingly found that use of a water-resistant coating, can be beneficial in alleviating such problems, which may be associated with prior art formulations.

### SUMMARY OF THE INVENTION

More particularly, there is now provided by the present invention a pharmaceutically acceptable oral formulation comprising core material which comprises a therapeutically effective amount of a 5-HT-receptor agonist, or a pharmaceutically acceptable salt, solvate or derivative thereof, which core material is provided with a substantially water resistant coating comprising one or more substantially water resistant materials.

As used herein, the term “therapeutically effective amount” means an amount of a 5-HT-receptor agonist which is capable of treating conditions in a human patient substantially as hereinafter described in greater detail. More particularly, the term “therapeutically effective amount” means an amount of a 5-HT-receptor agonist which is capable of treating migraine and related conditions. 5-HT-receptor agonists suitable for use in formulations according to the present invention include sumatriptan, zolmitriptan, naratriptan and rizatriptan, and pharmaceutically acceptable salts, solvates and derivatives thereof. In particular, it is preferred that a 5-HT-receptor agonist employed in a formulation according to the present invention comprises sumatriptan, or a pharmaceutically acceptable salt or solvate thereof, and particularly preferred is sumatriptan succinate.

The term “substantially water-resistant materials” as used herein can include, for example, waxes, and typically denotes coating materials which can provide a substantially water and moisture impermeable barrier around the core material. In this way, formulations according to the present invention can substantially prevent, or at least reduce, the possible degradation of a 5-HT-receptor agonist present in the core material of the formulations.

There is further provided by the present invention a pharmaceutically acceptable oral formulation comprising core material which comprises a therapeutically effective amount of a 5-HT-receptor agonist, or a pharmaceutically acceptable salt, solvate or derivative thereof, which core material is provided with a substantially water resistant coating comprising one or more substantially water resistant materials and wherein the 5-HT-receptor agonist is substantially free of degradation products associated with exposure of a 5-HT-receptor agonist to ambient moisture. The formulations according to the present invention are thus stable, can be easily swallowed and disintegrate rapidly further to administration. The wording “substantially free of degradation products” as used herein denotes minimal impurities, in particular [3-[2-(dimethylamino)ethyl]-2-[[3-[2-(dimethylamino)ethyl]-1H-indol-5-yl]methyl]-1H-indol-5-yl]-N-methylmethanesulphonamide, resulting further to degradation of a 5-HT-receptor agonist, as hereinafter described in greater detail.

More specifically, it can be seen that for a tablet formulation according to the present invention which includes 25 mg of sumatriptan succinate, under storage conditions of about 1 month at 25 EC and 60% relative humidity, less than about 0.6% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. More preferably, under storage conditions of about 1 month at 25 EC and 60% relative humidity, less than about 0.55% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. Even more preferably, under storage conditions of about 1 month at 25 EC and 60% relative humidity, about 0.5% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation.

Furthermore, for a tablet formulation according to the present invention which includes 25 mg of sumatriptan succinate, it is also preferably seen that under storage conditions of about 1 month at 40 EC and 75% relative humidity, less than about 0.65% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. More preferably, under storage conditions of about 1 month at 40 EC and 75% relative humidity, less than about 0.60% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. Even more preferably, under storage conditions of about 1 month at 40 EC and 75% relative humidity, about 0.55% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation.

It can also be seen that for a tablet formulation according to the present invention which includes 100 mg of sumatriptan succinate, under storage conditions of about 1 month at 25 EC and 60% relative humidity, less than about 0.60% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. More preferably, under storage conditions of about 1 month at 25 EC and 60% relative humidity, less than about 0.55% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. Even more preferably, under storage conditions of about 1 month at 25 EC and 60% relative humidity, about 0.50% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation.

Furthermore, for a tablet formulation according to the present invention which includes 100 mg of sumatriptan succinate, it is also preferably seen that under storage conditions of about 1 month at 40 EC and 75% relative humidity, less than about 0.65% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. More preferably, under storage conditions of about 1 month at 40 EC and 75% relative humidity, less than about 0.60% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. Even more preferably, under storage conditions of about 1 month at 40 EC and 75% relative humidity, about 0.55% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation.

Waxes suitable for use in a coating to be employed according to the present invention are water-resistant materials made up of various substances, including hydrocarbons (n-alkanes), ketones, diketones, primary and secondary alcohols, aldehydes, alkanoic acids, terpenes (squalene) and monoesters, all with long carbon chains (from 12-38 carbon atoms), which are solid over a wide temperature range (fusion point between 60-100° C.). More commonly, waxes are esters of a monohydric alcohol and a long chain acid.

Preferably a wax suitable for use in a formulation according to the present invention can be selected from the group consisting of beeswax, shellac, carnauba wax, spermaceti, lanolin, jojoba oil, candellila wax, ozocerite, Opaglos® 6000 P and the like. Most preferred waxes for use in the coating of a formulation according to the present invention are carnauba wax, beeswax or Opaglos® 6000 P.

### DETAILED DESCRIPTION OF THE INVENTION

In a first preferred embodiment of the present invention, there is thus provided a tablet formulation comprising core material which comprises a therapeutically effective amount of sumatriptan succinate, together with a substantially water resistant coating provided to said core material and comprising one or more waxes, or one or more wax derivatives, characterised in that said tablet formulation contains about 25 mg of sumatriptan succinate and under storage conditions of about 1 month at 25 EC and 60% relative humidity, less than about 0.60% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. More preferably, under storage conditions of about 1 month at 25 EC and 60% relative humidity, less than about 0.55% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. Even more preferably, under storage conditions of about 1 month at 25 EC and 60% relative humidity, about 0.50% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation.

Furthermore, for a tablet formulation according to the present invention which contains 25 mg of sumatriptan succinate as described above, it is also preferably seen that under storage conditions of about 1 month at 40 EC and 75% relative humidity, less than about 0.65% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. More preferably, under storage conditions of about 1 month at 40 EC and 75% relative humidity, less than about 0.60% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. Even more preferably, under storage conditions of about 1 month at 40 EC and 75% relative humidity, about 0.55% by weight of [3-[2-(dimethylamino)ethyl]-2-[[3-[2-(dimethylamino)ethyl]-1H-indl-5-yl]methyl]-1H-indol-5-yl]-N-methylmethanesulphonamide is present in the tablet formulation.

In a second preferred embodiment of the present invention, there is provided a tablet formulation comprising core material which comprises a therapeutically effective amount of sumatriptan succinate, together with a substantially water resistant coating provided to said core material and comprising one or more waxes, or one or more wax derivatives, characterised in that said tablet formulation contains about 100 mg of sumatriptan succinate and under storage conditions of about 1 month at 25 EC and 60% relative humidity, less than about 0.60% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. More preferably, under storage conditions of about 1 month at 25 EC and 60% relative humidity, less than about 0.55% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. Even more preferably, under storage conditions of about 1 month at 25 EC and 60% relative humidity, about 0.50% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation.

Furthermore, for a tablet formulation according to the present invention which includes 100 mg of sumatriptan succinate, it is also preferably seen that under storage conditions of about 1 month at 40 EC and 75% relative humidity, less than about 0.65% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. More preferably, under storage conditions of about 1 month at 40 EC and 75% relative humidity, less than about 0.60% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation. Even more preferably, under storage conditions of about 1 month at 40 EC and 75% relative humidity, about 0.50% by weight of [3-[2-(dimethylamino)ethyl]-2-[[3-[2-(dimethylamino)ethyl]-1H-indol-5-yl]methyl]-1H-indol-5-yl]-N-methylmethanesulphonamide is present in the tablet formulation.

There is further provided by the present invention use of one or more waxes, or one or more wax derivatives, to inhibit degradation of a 5-HT-receptor agonist susceptible to degradation on exposure to ambient moisture, wherein said one or more waxes, or one or more wax derivatives, provides a substantially water resistant coating to a core material comprising a 5-HT-receptor agonist, or a pharmaceutically acceptable salt, solvate or derivative thereof, of a pharmaceutically acceptable oral formulation. In particular, such use of one or more waxes, or one or more wax derivatives, inhibits formation of [3-[2-(dimethylamino)ethyl]-2-[[3-[2-(dimethylamino)ethyl]-1H-indol-5-yl]methyl]-1H-indol-5-yl]-N-methylmethanesulphonamide in an oral formulation as provided by the present invention.

There is also provided a method of substantially preventing the formation, in a pharmaceutically acceptable oral formulation, of degradation products, in particular [3-[2-(dimethylamino)ethyl]-2-[[3-[2-(dimethylamino)ethyl]-1H-indol-5-yl]methyl]-1H-indol-5-yl]-N-methylmethanesulphonamide, associated with exposure of a 5-HT-receptor agonist to ambient moisture, which method comprises providing core material comprising a 5-HT-receptor agonist, or a pharmaceutically acceptable salt, solvate or derivative thereof, with a substantially water resistant coating comprising one or more substantially water resistant materials.

For the above use and method as provided by the present invention, it is further preferred that the purity profile of a formulation as provided thereby is substantially as hereinbefore described.

It is preferred that the substantially water-resistant coating is directly applied to the core material, which can be desirable in providing physical stability and desired moisture protection. Core material present in a formulation according to the present invention is typically present in the form of a tablet, but may alternatively be provided in the form of granules.

Suitably the water-resistant coating of a formulation according to the present invention can further comprise one or more coating excipient materials, solvents for the waxes and plasticizers to coat solid formulations.

Suitably, therefore, the core material of a formulation as provided by the present invention can further comprise an excipient or bulking agent selected so as to provide the required properties for pharmaceutical usage, such as the required hardness, friability, disintegration time and the like. Preferred excipients can be selected from the group consisting of an alkali or alkali earth metal carbonate or bicarbonate (such as sodium carbonate, calcium carbonate, magnesium carbonate or sodium bicarbonate, preferably calcium carbonate) mannitol, dibasic calcium phosphate, xylitol, maltitol, sorbitol and erythritol, either present in anhydrous or hydrated form, or spray dried. The most preferred excipient can be mannitol, which may be anhydrous, hydrous or spray dried, or dibasic calcium phosphate, which is typically anhydrous, or calcium carbonate. The desired form of mannitol to be employed in a formulation according to the present invention should typically be selected based on desired pharmaceutical properties as referred to above, such as dissolution, content, uniformity, hardness, friability, disintegration time and the like. The appropriate choice of mannitol would be well known to one of ordinary skill in the art, in order to achieve the desired pharmaceutical properties of a pharmaceutical formulation according to present invention.

Suitably, the core material of a formulation according to the present invention further comprises a disintegrant. There are a variety of grades of disintegrants available, and the grade may be selected based on the acceptable batch variability. The preferred disintegrants include hydroxypropylcellulose, microcrystalline cellulose, croscarmellose sodium and other known disintegrants.

Suitable dry binders may also be employed using known methods. Such binders should be selected to provide satisfactory friability. A particularly preferred dry binder comprises hydroxypropylcellulose and/or microcrystalline cellulose. Other dry binders known in the art may also be selected.

An appropriate lubricant may also be employed, for example to prevent sticking of tablets to compression tooling. A preferred lubricant is magnesium stearate.

5-HT-receptor agonists, in particular sumatriptan, and salts, solvates and derivatives thereof, have therapeutic applicability for use in the treatment of migraine and associated conditions, for example cluster headache, chronic paroxysmal hemicrania, headache associated with vascular disorders, tension headache and paediatric migraine. There is further provided by the present invention, therefore, a method of treating a condition prevented, ameliorated or eliminated by administration of a 5-HT-receptor agonist, which method comprises administering to a human patient suffering from or susceptible to such a condition a therapeutically effective amount of a formulation according to the present invention substantially as hereinbefore described. In particular, the present invention provides a method of treating migraine and associated conditions, which method comprises administering to a human patient suffering from or susceptible to migraine and/or an associated condition, a therapeutically effective amount of a formulation according to the present invention substantially as hereinbefore described. The term “treatment” as used herein encompasses both prophylaxis, and the treatment of established conditions. The “treatment” can also include the management and care of a human patient for the purpose of combating, for example, migraine conditions as referred to above and can include the administration of a formulation according to present invention to prevent the onset of the symptoms or complications associated with such conditions, or alleviating or ameliorating the symptoms or complications associated with such conditions.

Substantially as hereinbefore described, sumatriptan is a preferred antimigraine compound for use according to the present invention and is effective over a wide dosage range, with the actual dose administered being dependent on the condition being treated and also the patient. Single doses of 25, 50, or 100 mg of sumatriptan succinate tablets have been shown to be effective for the acute treatment of migraine in adults. If a headache returns, or the patient has a partial response to the initial dose, the dose may be repeated after 2 hours, but should not exceed a total daily dose of 200 mg.

A preferred formulation according to the present invention is an oral formulation comprising core material comprising about 20 to 100 mg of sumatriptan succinate as an active ingredient, wherein the core material is coated with a substantially water resistant coating comprising one or more substantially water resistant materials, such as one or more waxes, or one or more wax derivatives. More particularly, core material as present in a preferred formulation according to the present invention typically comprises sumatriptan succinate, mannitol or dibasic calcium phosphate or calcium carbonate, hypromellose and/or microcrystalline cellulose, croscarmellose sodium and magnesium stearate. In certain embodiments, it is preferred that the core material comprises sumatriptan succinate, mannitol, hypromellose and/or microcrystalline cellulose, croscarmellose sodium and magnesium stearate. Alternatively, it can be preferred that the core material as present in a preferred formulation according to the present invention typically comprises sumatriptan succinate, dibasic calcium phosphate, hypromellose and/or microcrystalline cellulose, croscarmellose sodium and magnesium stearate. Alternatively, it can be preferred that the core material as present in a preferred formulation according to the present invention typically comprises sumatriptan succinate, calcium carbonate, hypromellose and/or microcrystalline cellulose, croscarmellose sodium and magnesium stearate. Especially preferably, core material as present in a preferred formulation according to the present invention typically comprises about 20 to 55% w/w sumatriptan succinate, about 20 to 50% w/w mannitol or dibasic calcium phosphate or calcium carbonate, about 1 to 10% w/w hypromellose and/or microcrystalline cellulose, about 1 to 5% w/w croscarmellose sodium and about 0.5 to 2% w/w magnesium stearate. In certain embodiments, core material as present in a preferred formulation according to the present invention typically comprises about 20 to 55% w/w sumatriptan succinate, about 20 to 50% w/w mannitol, about 1 to 10% w/w hypromellose and/or microcrystalline cellulose, about 1 to 5% w/w croscarmellose sodium and about 0.5 to 2% w/w magnesium stearate. In alternative embodiments, core material as present in a preferred formulation according to the present invention typically comprises about 20 to 55% w/w sumatriptan succinate, about 20 to 50% w/w dibasic calcium phosphate, about 1 to 10% w/w hypromellose and/or microcrystalline cellulose, about 1 to 5% w/w croscarmellose sodium and about 0.5 to 2% w/w magnesium stearate. In alternative embodiments, core material as present in a preferred formulation according to the present invention typically comprises about 20 to 55% w/w sumatriptan succinate, about 20 to 50% w/w calcium carbonate, about 1 to 10% w/w hypromellose and/or microcrystalline cellulose, about 1 to 5% w/w croscarmellose sodium and about 0.5 to 2% w/w magnesium stearate.

There is still further provided by the present invention a process of preparing a pharmaceutically acceptable oral formulation substantially as hereinbefore described, which process comprises providing core material which comprises a therapeutically effective amount of a 5-HT-receptor agonist, or a pharmaceutically acceptable salt, solvate or derivative thereof, and providing the core material with a substantially water resistant coating comprising one or more substantially water resistant materials.

Direct compression processes, dry granulated processes, wet granulation processes or fluidized bed processing technology could provide suitable processes for preparing pharmaceutical oral formulations according to the present invention. The present invention further provides, therefore, a process of preparing a pharmaceutical formulation substantially as hereinbefore described, which process may comprise wet granulation or direct compression techniques.

### EXAMPLES

The present invention will now be illustrated by the following examples, which do not limit the scope of the invention in anyway.

### Example-I

Sumatriptan succinate was blended with a bulking agent and dry binders, namely mannitol, hypromellose, microcrystalline cellulose and croscarmellose sodium, and then mixed with magnesium stearate and compressed. The core so obtained was coated with water-resistant materials suitable for use according to the present invention, namely a wax dissolved in mixture of solvents, and more specifically carnauba wax dissolved in a mixture of isopropyl alcohol and methylene chloride.

| Sr. No. | Ingredients | Quantity (mg/tablet) |
|---|---|---|
| 1. | Sumatriptan succinate | 35.00 |
| | equivalent to sumatriptan | |
| 2. | Mannitol | 30.25 |
| 3. | Croscarmellose sodium | 3.00 |
| 4. | Hypromellose | 2.00 |
| 5. | Avicel PH112 | 5.00 |
| 6. | Magnesium Stearate | 0.75 |
| | Coating | |
| 7. | Carnauba wax | 4.00 |
| 8. | Isopropyl Alcohol | q.s. |
| 9. | Methylene Chloride | q.s. |

### Example-II

Sumatriptan succinate was blended with a bulking agent and dry binders, namely calcium carbonate, hypromellose, microcrystalline cellulose and croscarmellose sodium, and then mixed with magnesium stearate and compressed. The core so obtained was coated with water-resistant materials suitable for use according to the present invention, namely a wax dissolved in mixture of solvents, and more specifically carnauba wax dissolved in a mixture of isopropyl alcohol and methylene chloride.

| Sr. No. | Ingredients | Quantity (mg/tablet) |
|---|---|---|
| 1. | Sumatriptan succinate | 35.00 |
| | equivalent to sumatriptan | |
| 2. | Calcium Carbonate | 30.25 |
| 3. | Croscarmellose sodium | 3.00 |
| 4. | Hypromellose | 2.00 |
| 5. | Purified water | q.s. |
| 6. | Avicel PH112 | 5.00 |
| 7. | Magnesium Stearate | 0.75 |
| | Coating | |
| 8. | Carnauba wax | 4.00 |
| 9. | Isopropyl Alcohol | q.s. |
| 10. | Methylene Chloride | q.s. |

### Example-III

The procedure described in Example 1 was repeated for the following ingredients.

| Sr. No. | Ingredients | Quantity (mg/tablet) |
|---|---|---|
| 1. | Sumatriptan succinate | 35.00 |
| | equivalent to sumatriptan | |
| 2. | Mannitol | 30.25 |
| 3. | Croscarmellose sodium | 3.00 |
| 4. | Hypromellose | 2.00 |
| 5. | Purified water | q.s. |
| 6. | Avicel PH112 | 5.00 |
| 7. | Magnesium Stearate | 0.75 |
| | Coating | |
| 8. | Opaglos ® 6000 P | 1.00 |
| 9. | Isopropyl alcohol | q.s. |

### Example IV

The procedure described in Example 1 was repeated for the following ingredients.

| Sr. No. | Ingredients | Quantity (mg/tablet) |
|---|---|---|
| 1. | Sumatriptan succinate | 35.00 |
| | equivalent to sumatriptan | |
| 2. | Mannitol | 30.25 |
| 3. | Croscarmellose sodium | 3.00 |
| 4. | Hypromellose | 2.00 |
| 5. | Purified water | q.s. |
| 6. | Avicel PH112 | 5.00 |
| 7. | Magnesium Stearate | 0.75 |
| | Coating | |
| 8. | Carnauba wax | 2.00 |
| 9. | Bees wax | 4.00 |
| 10. | Chloroform | q.s. |

### Example-V

Sumatriptan succinate was mixed with DCP anhydrous. The remaining excipients were added to resulting drug mix, followed by lubricating with magnesium stearate and compressing in a suitable tooling. The resulting tablet cores were coated with Opaglos® 6000 P.

| Sr. No. | Ingredients | Quantity (mg/tablet) |
|---|---|---|
| 1. | Sumatriptan succinate | 140.00 |
| 2. | Dibasic calcium phosphate DCP | 86.70 |
| | anhydrous | |
| 3. | Croscarmellose sodium (Ac-di-sol) | 24.00 |
| 4. | Sodium Carbonate | 20.00 |
| 5. | Hydroxypropylmethylcellulose | 4.80 |
| | (3cps) | |
| 6. | Microcrystalline cellulose (Avicel | 20.00 |
| | pH112) | |
| 7. | Magnesium Stearate | 4.50 |
| 8. | Opaglos ® 6000 P | 4.00 |
| 9. | IPA | q.s. |
| | Total | 300.00 |

### Example-VI

The following Table shows the impurity profile of a wax coated sumatriptan succinate tablet (25 mg) as provided by the present invention, under storage conditions of (i) 1 month at 25 EC and 60% relative humidity and (ii) 1 month at 40 EC and 75% relative humidity, and a comparison with the corresponding uncoated sumatriptan tablet.

| | Uncoated IM Exposure | | | Wax coated 1M Exposure | | |
|---|---|---|---|---|---|---|
| | | 25EC/ | 40EC/ | | 25EC/ | 40EC/ |
| Impurity | Initial | 60% RH | 75% RH | Initial | 60% RH | 75% RH |
| [3-[2-(dimethylamino)ethyl]- | 0.21% | 0.21% | 0.33% | 0.15% | 0.16% | 0.20% |
| 2-[[3-[2-(dimethylamino)ethyl]- | | | | | | |
| 1H-indol-5-yl]methyl]-1H- | | | | | | |
| indol-5-yl]-N-methylmethane | | | | | | |
| sulphonamide | | | | | | |

The following Table shows the impurity profile of a wax coated sumatriptan succinate tablet (100 mg) as provided by the present invention, under storage conditions of (i) 1 month at 25 EC and 60% relative humidity and (ii) 1 month at 40 EC and 75% relative humidity, and a comparison with the corresponding uncoated sumatriptan tablet.

| | Uncoated IM Exposure | | | Wax coated 1M Exposure | | |
|---|---|---|---|---|---|---|
| | | 25EC/ | 40EC/ | | 25EC/ | 40EC/ |
| Impurity | Initial | 60% RH | 75% RH | Initial | 60% RH | 75% RH |
| [3-[2-(dimethylamino)ethyl]- | 0.21% | 0.27% | 0.38% | 0.17% | 0.18% | 0.19% |
| 2-[[3-[2-(dimethylamino)ethyl]- | | | | | | |
| 1H-indol-5-yl]methyl]-1H- | | | | | | |
| indol-5-yl]-N-methylmethane | | | | | | |
| sulphonamide | | | | | | |

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The discussion of a reference in the Description of Related Art is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

What is claimed is:

1. A pharmaceutically acceptable oral formulation comprising core material which comprises a therapeutically effective amount of a triptan 5-HT-receptor agonist, or a pharmaceutically acceptable salt or solvate thereof, which core material is provided with a substantially water resistant coating comprising one or more substantially water resistant materials, wherein said one or more substantially water resistant materials comprise one or more waxes, and wherein the coating provides a substantially water impermeable and a substantially moisture impermeable barrier around the core, wherein said wax is selected from group consisting of beeswax, shellac, carnauba wax, spermaceti, lanolin, jojoba oil, candellila wax and ozocerite.

2. The pharmaceutically acceptable oral formulation according to claim 1, wherein said 5-HT-receptor agonist is selected from the group consisting of sumatriptan, zolmitriptan, naratriptan and rizatriptan, and pharmaceutically acceptable salts, solvates and derivatives thereof.

3. The pharmaceutically acceptable oral formulation according to claim 2, wherein said 5-HT-receptor agonist is sumatriptan, or a pharmaceutically acceptable salt or solvate thereof.

4. The pharmaceutically acceptable oral formulation according to claim 3, wherein said 5-HT-receptor agonist is sumatriptan succinate.

5. The pharmaceutically acceptable oral formulation according to claim 1, wherein said wax is selected from the group consisting of carnauba wax and beeswax.

6. The pharmaceutically acceptable oral formulation according to claim 1, wherein said substantially water-resistant coating further comprises one or more coating excipient materials, solvents for the waxes and plasticizers to coat solid formulations.

7. The pharmaceutically acceptable oral formulation according to claim 1, wherein said substantially water-resistant coating is directly applied to the core material.

8. The pharmaceutically acceptable oral formulation according to claim 1, wherein said core material is present in the form of a tablet.

9. The pharmaceutically acceptable oral formulation according to claim 1, wherein said core material is present in the form of granules.

10. The pharmaceutically acceptable oral formulation according to claim 1, wherein the core material comprises about 20 to 100 mg of sumatriptan succinate as an active ingredient.

11. The pharmaceutically acceptable oral formulation according to claim 10, wherein said core material comprises about 20 to 55% w/w sumatriptan succinate, about 20 to 50% w/w mannitaol, about 1 to 10% w/w hypromellose and/or microcrystalline cellulose, about 1 to 5% w/w croscarmellose sodium and about 0.5 to 2% w/w magnesium stearate.

12. A method comprising utilizing a substantially water and moisture impermeable wax coating to inhibit degradation of a triptan 5-HT-receptor agonist susceptible to degradation on exposure to ambient moisture, wherein said coating provides a substantially water resistant coating to a core material comprising the triptan 5-HT-receptor agonist, or a pharmaceutically acceptable salt or solvate, of a pharmaceutically acceptable oral formulation wherein said wax is selected from group consisting of beeswax, shellac, carnauba wax, spermaceti, lanolin, jojoba oil, candellila wax and ozocerite.

13. The method according to claim 12, wherein said 5-HT-receptor agonist is selected from the group consisting of sumatriptan, zolmitriptan, naratriptan and rizatriptan, and pharmaceutically acceptable salts, solvates and derivatives thereof.

14. The method according to claim 13, wherein said 5-HT-receptor agonist is sumatriptan, or a pharmaceutically acceptable salt or solvate thereof.

15. The method according to claim 14, wherein said 5-HT-receptor agonist is sumatriptan succinate.

16. The method according to claim 12, wherein said wax is selected from the group consisting carnauba wax and beeswax.

17. A process for preparing a pharmaceutically acceptable oral formulation according to claim 1, which process comprises providing core material which comprises a therapeutically effective amount of a 5-HT-receptor agonist, or a pharmaceutically acceptable salt, solvate or derivative thereof, and providing the core material with a substantially water resistant coating comprising one or more substantially water resistant materials, wherein said one or more substantially water resistant materials comprise one or more waxes, or one or more wax derivatives.

18. The process according to claim 17 which employs wet granulation or direct compression techniques.

19. The process according to claim 17, wherein said 5-HT-receptor agonist is selected from the group consisting of sumatriptan, zolmitriptan, naratriptan and rizatriptan, and pharmaceutically acceptable salts, solvates and derivatives thereof and wherein said wax is selected from the group consisting carnauba wax and beeswax.

20. The process according to claim 17, wherein said 5-HT-receptor agonist is sumatriptan, or a pharmaceutically acceptable salt or solvate thereof and wherein said wax is selected from the group consisting carnauba wax and beeswax.
